(19) United States
(12) Patent Application Publication
(10) Pub. No.: US 2011/0245614 A1
(43) Pub. Date: Oct. 6, 2011

(54) ENDOSCOPIC APPARATUS AND METHOD OF MANUFACTURING THE SAME

(75) Inventor: Shigeru NAKAMURA, Ashigarakami-gun (JP)

(73) Assignee: FUJIFILM CORPORATION, Tokyo (JP)

(21) Appl. No.: 13/017,836

(22) Filed: Jan. 31, 2011

(30) Foreign Application Priority Data
Mar. 31, 2010 (JP) 2010-084550

(51) Int. Cl. A61B 1/00; B32B 37/12; B65H 81/00

(52) U.S. Cl. 600/140; 156/275.5; 156/184

(57) ABSTRACT

The present invention provides an endoscopic apparatus including a partial structure, wherein the partial structure includes:
a metallic member;
a polymer layer that contains a polymer including a vinylidene fluoride unit; and
an epoxy resin layer disposed on the polymer layer,
and wherein the polymer layer and the epoxy resin layer are disposed in this order on a surface of at least a part of the metallic member.

### CROSS-REFERENCE TO RELATED APPLICATION

This application claims priority under 35 USC 119 from Japanese Patent Application No. 2010-084550, filed on Mar. 31, 2010, the disclosure of which is incorporated by reference herein.

### BACKGROUND

1. Field of the Invention

The present invention relates to an endoscopic apparatus and a method of manufacturing the same, and more particularly, to an endoscopic apparatus having a partial structure which is useful for the formation of a unit that covers a metallic member with another member, and a joint area that fixes to the metallic member a fixable unit which is to be fixed to the metallic member, and a method of manufacturing the endoscopic apparatus.

2. Related Art

Medical endoscopes perform an observation of organs and the like by having the insertion unit inserted into a body cavity, or various treatments or remedies by using a treatment tool inserted into a treatment tool insert-through channel of the endoscope.

Therefore, when an endoscope that has once been used is to be reused in another patient, it is required to disinfect and sterilize the endoscope after the completion of examination and treatment, in order to prevent the infection between patients mediated by the endoscope. Disinfection or sterilization is achieved by methods of using a disinfectant liquid, ethylene oxide gas, formalin gas, hydrogen peroxide gas plasma, ozone, a sterilized autoclave which uses high temperature high pressure steam, and the like.

In recent years, it is desired to make the flexible tube of endoscopes to have a finer diameter, in order to reduce the burden to patients. Thus, in order to pack various members in a narrow space for the purpose of imparting high functions, coupling is effectively achieved not by using bulky mechanical coupling members such as bolts and screws, but by using an adhesive. Many of currently used endoscopes use adhesives in the connection between members.

For example, the flexible tube for insertion unit of an endoscope, or the curved portion connected to the front tip of the flexible tube is coated with a soft casing having a flexible tube shape, in order to protect the member or to reduce the adverse effects on human body. However, at the areas to which such a soft casing is fixed, the end portion of the soft casing is fixed by a thread winding method, and the thread winding structure is coated with an epoxy adhesive layer, so as to prevent fraying of the thread and also to realize a smooth appearance. In addition to that, adhesives are also used in the connection of a forceps tube via forceps for treatment, the adhesive sealing of the window material at the front tip, and the like.

Furthermore, even in those rigid endoscopic apparatuses provided with flexible members, coupling is similarly achieved by an adhesive at the coupled section between a soft casing tube in the flexible member and a stainless steel tube of the main body.

A hydrogen peroxide plasma method, which is a representative technique for high-level sterilization treatment for endoscopes, is a method of decomposing hydrogen peroxide by means of plasma to generate active hydroxy radicals, and thereby achieving sterilization. Furthermore, a disinfection and sterilization method that has been widely popularized involves the use of an autoclave which sterilizes an endoscope with high temperature high pressure vapor. All of these methods have numerous merits such as high reliability in the sterilization effect, lack of residual toxicity, and low running cost; however, under the relevant environment, there is a problem that the impact on the endoscopic apparatus, and particularly on the soft member, is significant. The inventor of the invention conducted an investigation, and as a result, it was clearly found that a metallic member containing iron such as stainless steel, which is used in rigid endoscopic apparatuses, is subjected to an accelerated oxidizing action of hydrogen peroxide due to the iron ions released by the action of an acid resulting from a hydrogen peroxide plasma treatment, and causes noticeable damage to the adjacent resin material. Such an accelerated oxidizing action causes deterioration of organic materials under the conditions more severe than usual, and there is a concern about the possible situation in which the water-tight structure of an endoscope is destroyed by the deterioration of organic materials at the fixing unit, and the function of the water-tight structure is lost.

For the adhesive to be used at the joint section and joint portion of metallic members, an epoxy resin adhesive is generally used from the viewpoint of strength.

A cured product of an epoxy resin adhesive has its heat resistance and chemical resistance enhanced by increasing the crosslinking density, but on the other hand, there is a problem that if the crosslinking density is increased, the brittleness of the epoxy resin layer becomes poor, so that the adhesive power decreases when it is attempted to fix a flexible member or the like.

Therefore, conventionally, an epoxy material that is used as an adhesive is blended with a good balance between required performance items that are contradictory to each other, so that the required adhesiveness is secured in substitution of a decrease in heat resistance and chemical resistance, by regulating the type of a curing agent, and the mixing ratio of the amounts of the epoxy main agent and the curing agent. However, with regard to the coupled section which is formed by protecting the thread winding area of a soft tube using an adhesive of such blend, it is desirable to have sufficient durability against a treatment using various high-potential disinfectant solutions, and particularly against autoclave sterilization and hydrogen peroxide plasma sterilization treatment.

In regard to such a problem, there has been suggested an attempt to solve the problem by blending a rigid epoxy resin with rubber or resin particles to impart flexibility (see, for example, Japanese Patent Application Laid-Open (JP-A) No. 2006-218102). However, even with this method, there is room for an improvement in the aspect that high adhesiveness may be realized without reducing the chemical resistance and heat resistance of the rigid epoxy resin.

Particularly, deterioration is noticeable at the area where an epoxy adhesive is directly used on the surface of a metallic member, and there has been a demand for a technique for enhancing durability, in addition to the selection of an adequate adhesive.

### SUMMARY

The present invention has been made in view of the above circumstances and provides an endoscopic apparatus comprising a partial structure, wherein the partial structure includes:

a metallic member;

a polymer layer that contains a polymer including a vinylidene fluoride unit; and

an epoxy resin layer disposed on the polymer layer,

and wherein the polymer layer and the epoxy resin layer are disposed in this order on a surface of at least a part of the metallic member.

### BRIEF DESCRIPTION OF THE DRAWINGS

Exemplary embodiments of the present invention will be described in detail, based on the following figures, wherein:

FIG. 1 is an overall configuration diagram for an ultrasonic endoscopic apparatus according to the present exemplary embodiment; and

FIG. 2 is a partial schematic cross-section diagram displaying a magnified view of the vicinity of a joint unit of a metallic member in the ultrasonic endoscopic apparatus shown in FIG. 1.

### DETAILED DESCRIPTION OF THE INVENTION

Hereinafter, exemplary embodiments of the invention will be described with reference to the attached drawings.

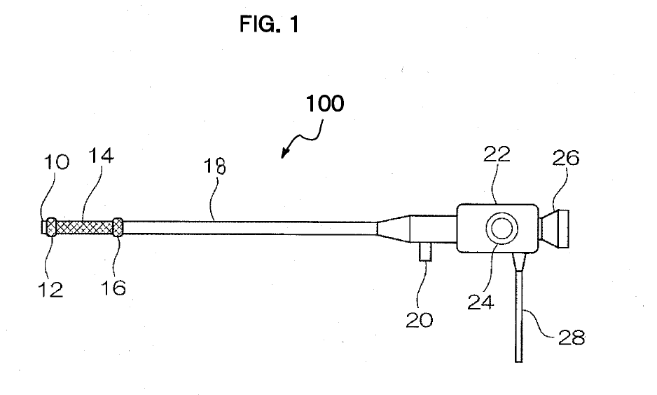

FIG. 1 is an external appearance diagram of an endoscope according to a first exemplary embodiment of the invention.

An endoscope 100 according to the exemplary embodiment includes an operation unit 22; an insertion unit which is extended from the operation unit 22, has a channel hole formed inside, and includes a front tip section 10, a curved section 14 and a rigid section 18; and a light guide (LG) soft section 28 which is connected to the operation unit 22. The light guide (LG) soft section 28 is connected detachably from and attachably to, although not depicted, a light source apparatus via a connector, a relay code soft section or the like.

The operation unit 22 is provided with a forceps opening 20 into which a treatment tool such as forceps is inserted, and is also provided with a vertical angle lever 24. There is provided, on the opposite side of the side of the operation unit 22 where the insertion unit is extended, an ocular lens section 26 for observing endoscopic observation images. Furthermore, the operation unit 22 may also be provided with an FR switch, a suction switch unit or the like, as necessary.

The insertion unit is composed of a rigid section 18, a curved section 14 and a front tip section 10, and the curved section 14 is remotely operated to curve by operating the vertical angle lever 24 installed in the operation unit 22, so that the front tip section 10 is head toward a desired direction.

Between the deformable curved section 14 and the front tip section 10, there is provided a joint section (A) 12 that joins and fixes the two units, and between the curved section 14 and the rigid section 18, there is provided a joint section (B) 16 that joins and fixes the two units.

The front tip section 10 in the insertion unit is provided with an illumination window provided with a lens for illumination and an observation window provided with an object lens, which together compose an endoscopic observation instrument that is not depicted. The illumination window faces the exit end surface of a light guide formed from a bundle of optic fibers, and this light guide is configured to be extended into the light guide soft section 28 via the operation unit 22 and to be connected to a light source device. Furthermore, the imaging position of the object lens in the observation window 31 faces the incident end of an image guide formed from a bundle of optical fibers, and the other end of this image guide is extended into the ocular lens section 26 and is disposed so as to confront the ocular lens provided in the ocular lens section 26.

In addition to the insert-through members described above, the insertion unit is provided with a treatment tool insert-through channel 34 (not shown) for inserting through forceps and other treatment tools, so that the forceps and other treatment tools may be inserted into a body cavity from the forceps opening 20 mentioned above through the treatment tool insert-through channel. Then, the endoscope is operated in a state in which the forceps and other treatment tools are projected only for a predetermined length from the front tip of the treatment tool insert-through channel.

Here, the joint section (B) 16 between the rigid section 18 and the curved section 14 in the insertion unit corresponds to the joint portion according to the invention.

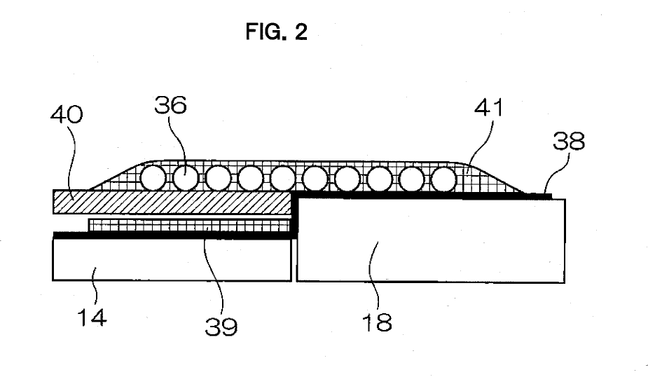

FIG. 2 is a partial cross-section diagram showing an aspect showing the joint state of the joint section (B) 16.

The rigid section 18 made of SUS and the curved section 14 are disposed so as to abut each other. A layer 38 containing a polymer which includes a vinylidene fluoride unit is provided to a surface of at least a part of the rigid section 18 and a surface of at least a part of the curved section 14 at or around the joint between the rigid section 18 and the curved section 14. A flexible casing 40 having a tube-like shape is adhered to the surface of the curved section 14, with the layer 38 and an epoxy resin layer 39 interposed therebetween. The end portion of the curved section 14 covered with the flexible casing 40 and the end portion of the rigid section 18 are disposed so as to abut each other. The outer peripheral surface of the curved section 14 covered with the flexible casing 40 and the outer peripheral surface of the rigid section 18 are fastened by winding with a thread 36 to form a thread winding structure, so that the curved section 14 covered with the flexible casing 40 is fixed to the rigid section 18. In the region over the flexible casing 40 and the rigid section 18, where the thread winding structure formed by fastening by winding with the thread 36 is present, an epoxy resin layer 41 to which a composition containing an epoxy resin is applied is formed on the outer periphery of the region so as to cover the thread 36. The epoxy resin layer prevents fraying or breaking of the thread 36 and covers the outside of the thread winding structure to form a smooth curved surface.

According to the invention, the rigid section 18 and the curved section 14, each of which is a metallic member, and the epoxy resin layers 39 and 41 are joined, with the layer 38 containing a polymer which includes a vinylidene fluoride unit interposed therebetween. The layer 38 containing a polymer which includes a vinylidene fluoride unit protects the surface of the metallic member and enhanced the adhesiveness between the outer periphery epoxy resin layers 39 and 41, and the metallic member. The layer 38 containing a polymer which includes a vinylidene fluoride unit functions to protect the interface between the flexible casing 40 and the curved section 14, so that deterioration of the epoxy resin layers 39 and 41 under the severe sterilization conditions such as autoclave or plasma, particularly due to the release of metal ions by hydrogen peroxide, is effectively suppressed, and deterioration of the metallic member and a decrease in the strength of the joint section, as well as exposure and breakage of the thread 36, which cause deterioration of the epoxy resin layer 41, are also suppressed.

Next, various materials for forming the partial structures such as described above, together with the method for production thereof, will be explained in detail.

<Layer Containing Polymer which Includes Vinylidene Fluoride Unit (Particular Polymer Layer)>

First, the materials that constitute the layer 38 containing a polymer which includes a vinylidene fluoride unit (hereinafter, appropriately referred to as particular polymer layer) will be explained.

In order to form the particular polymer layer 38 for the partial structure such as shown in FIG. 2, there may be employed a method of applying a composition for the formation of a particular polymer layer, which contains a polymer including a vinylidene fluoride unit (hereinafter, appropriately referred to as particular polymer) in a region where the particular polymer layer is to be formed on the surface of the metallic member (hereinafter, arbitrarily refers to rigid section 18 and/or curved section 14), and drying the composition.

The coating liquid composition containing a polymer which includes a vinylidene fluoride unit contains a polymer which includes a vinylidene fluoride unit as a main component.

There are no particular limitations on the particular polymer as long as the particular polymer has a vinylidene fluoride unit as a polymerization component, but from the viewpoint of the effects of the invention, the particular polymer preferably includes the vinylidene fluoride unit in an amount of 40% by mole or more, and more preferably in the range of from 50% by mole to 90% by mole.

When the content of the vinylidene fluoride unit is in the range described above, a uniform polymer layer is formed, and a sufficient adhesive power to the adjacent epoxy resin layer is obtained. In addition, the particular polymer may be composed only of a vinylidene fluoride unit, but if the content of the vinylidene fluoride unit is greater than 90% by mole, it tends to be difficult to form a uniform polymer layer. From such a viewpoint, it may be a preferred embodiment that the particular polymer is a copolymer including a unit other than the vinylidene fluoride unit in order to enhance coatability.

From the viewpoint of enhancing the oxidation resistance of the particular polymer and the liquid component penetration suppressing ability, it is preferable that the particular polymer include, as a copolymerization component, a fluorine-containing unit such as a monomer having a fluoroalkyl group or a fluorovinyl group.

Preferred examples of the particular polymer include a polymer constituted to include 40% by mole to 90% by mole of a vinylidene fluoride unit, and 10% by mole to 50% by mole of a fluoroalkylene group-containing unit or 10% by mole to 50% by mole of a fluorovinyl ether unit; and a polymer constituted to include 40% by mole to 90% by mole of a vinylidene fluoride unit, and 10% by mole to 30% by mole of a fluoroalkylene group-containing unit, and 10% by mole to 30% by mole of a fluorovinyl ether unit.

Examples of the fluoroalkylene group-containing unit include hexafluoropropylene and trifluoroethylene.

Examples of the fluorovinyl ether unit include perfluorovinyl ether.

Examples of the particular polymer that may be used in the invention include, for example, copolymers of vinylidene fluoride and one or plural copolymerization components selected from hexafluoropropylene, trifluoroethylene and perfluorovinyl ether.

A graft polymer obtained by graft copolymerizing polyvinylidene fluoride to a copolymer having vinylidene fluoride and chlorotrifluoroethylene as main components, and the like may also be used as the particular polymer according to the invention.

Specific examples of the particular polymer that may be used in the invention include fluororubbers. The particular polymer may be obtained as commercially available products, and examples thereof include G-902 (trade name, manufactured by Daikin Industries, Ltd.; including 40% by mole to 90% by mole of a vinylidene fluoride unit), and CEFRAL SOFT (trade name, manufactured by Central Glass Co., Ltd.; including 40% by mole to 90% by mole of a vinylidene fluoride unit). These may be suitably used as the particular polymer according to the invention.

When the particular polymer is cured to form a polymer layer, it is preferable that the polymer layer contain a crosslinked structure, from the viewpoint of enhancing durability. Therefore, it is also a preferred embodiment to use a particular polymer that is capable of forming a crosslinked structure through energy application such as ultraviolet radiation or heating during curing, or to use in combination an additive that is capable of forming a crosslinked structure in the composition for the formation of a polymer layer.

One preferred aspect may be a particular polymer having a functional group that is capable of forming peroxide crosslinking. Preferred examples of such a particular polymer include crosslinkable particular polymers having a functional group that is capable of forming a crosslinked structure (crosslinkable group), including a highly active iodine atom or a bromine atom. When a crosslinkable particular polymer is used, it is made possible to achieve thermal crosslinking in the presence of an organic peroxide and a co-crosslinking agent.

In addition, since the particular polymer may be subjected to radiation crosslinking or electron beam crosslinking in addition to the thermal crosslinking, even when a particular polymer that does not have a special crosslinking group is used, a crosslinked structure may be formed within the polymer layer by irradiating the polymer layer with a radiation or an electron beam.

When it is intended to form a layer containing the polymer which includes a vinylidene fluoride unit according to the invention, a single kind of the particular polymer may be used, or plural kinds may also be used in mixture.

Upon the formation of the particular polymer layer according to the invention, various additives may be used in addition to the particular polymer, to an extent of not impairing the effects of the invention.

In the case of using the particular polymer having a crosslinkable group as the particular polymer, it is preferable that the particular polymer contain an organic peroxide and a co-crosslinking agent.

As the organic peroxide, a compound which is usually used in the crosslinking of a fluororubber may be used.

It is also preferable that the composition for the formation of a particular polymer layer contain a silane coupling agent having a vinyl group. When the composition contains a silane coupling agent, vulcanizing adhesion with an adjacent metallic member may be achieved, and this is also useful for an enhancement of adhesiveness of a metallic member and the particular polymer layer.

The silane coupling agent that may be used in the invention is preferably a silane coupling agent having a vinyl group, and examples thereof include trialkoxysilanes such as vinyltrimethoxysilane and vinyltriethoxysilane, and oligomer type silane coupling agents obtained by condensing these trialkoxysilanes with an alkoxysilane such as triethoxysilane.

The amount of addition of the silane coupling agent is preferably about 1% by mass to 5% by mass based on the resin.

The particular polymer layer may also contain a filler such as carbon black, for an enhancement of the film strength.

The formation of the particular polymer layer may be carried out by dissolving or dispersing the particular polymer and the aforementioned additives that are used as desired, in a volatile solvent to prepare a composition for the formation of a particular polymer layer, applying this composition on the surface of a metallic member, and drying the composition.

The solvent to be used in the preparation of the composition for the formation of a particular polymer layer may be any solvent that is capable of uniformly dissolving the particular polymer, but preferred examples include ketone-based solvents such as acetone, methyl ethyl ketone and cyclohexanone; and ester-based solvent such as ethyl acetate and butyl acetate. These solvents are preferably used singly or as mixtures of plural kinds

The solids concentration in the composition for the formation of a particular polymer layer is preferably in the range of 0.1% by mass to 5% by mass, from the viewpoint of the ability of uniform application.

There are no particular limitations on the method of applying the composition for the formation of a particular polymer layer, and the application may be carried out by any known method. For example, immersion in the composition, brush coating, spray coating and the like may be employed.

Examples of the method of drying and curing the composition layer after application include a method of heating the composition in a non-contact manner such as blowing hot air or placing the composition layer in a heating zone; a method of heating the composition layer by allowing the layer to face a heater or the like from the metallic member side; and a method of drying in a vacuum. Among them, a method of treating the composition in a hot nitrogen gas stream at 100° C. to 180° C., and preferably approximately 150° C., is preferred. The treatment time is preferably about 1 to 5 minutes.

The thickness after drying of the particular polymer layer is preferably in the range of 0.1 μm to 50 μm, and more preferably in the range of 0.5 μm to 10 μm, from the viewpoint of the effects of the invention. When the thickness is in the above range, a film having excellent durability is formed, so that the film may satisfactorily protect the surface of a metallic member from steam, plasma, hydrogen peroxide or the like, and a decrease in durability due to the cohesive failure of the film is also suppressed.

For the purpose of enhancing the adhesiveness between the particular polymer layer and a metallic member, a silane coupling agent may be applied in advance on the surface of the metallic member. When an alcohol solution of a silane coupling agent at a concentration of 0.1% by mass to 1% by mass is prepared and applied on the surface of a metallic member, and then applying a coating liquid for the formation of a particular polymer layer to thereby provide a particular polymer layer, the metallic member and the particular polymer layer form a crosslinked structure by the function of the silane coupling agent, and thus adhesiveness is enhanced.

(Epoxy Resin Layer)

Subsequently, an epoxy resin layer is formed on the surface of the particular polymer layer. In the formation of the epoxy resin layer, a liquid state epoxy resin is preferably used.

Examples of the epoxy resin that may be suitably used in the invention include a bisphenol A type epoxy resin, a bisphenol F type epoxy resin, a bisphenol AD type epoxy resin, and a novolac type epoxy resin, and the epoxy resin is appropriately selected from the group of these.

There are no particular limitations on the curing agent that is used in combination with the epoxy resin as long as a cured product having the desired flexibility, specifically, the desired elastic modulus, is obtained. Any curing agent selected from known curing agents for epoxy resin-based adhesives, including any amine-based curing agents such as linear aliphatic amines, cyclic aliphatic amines, aromatic amines, and nitrogen-containing aromatics such as imidazole compounds, and amidoamine curing agents, may be used singly, or two or more kinds may be used in combination.

In regard to the mixing amount of the curing agent with respect to the epoxy resin, it is preferable to allow the epoxy groups and the amino groups to react in an amount close to an equivalent. In the present specification, the molecular weight per functionality of an epoxy resin is referred to as epoxy equivalent, and in the case of using an amine-based curing agent as curing agent, the amine equivalent is referred to as active hydrogen equivalent. The mixing ratio indicates an optimal range defined based on the theoretical mixing ratio calculated from the epoxy equivalent and the amine equivalent, as well as general properties such as adhesive strength.

In the composition for forming an epoxy resin layer, various additives such as a reactive diluent, a volatile solvent, a coupling agent and a filler may be used together in addition to the epoxy resin and the curing agent according to the purpose, to an extent of not impairing the effects of the invention.

The reactive diluent is added for the purpose of enhancing coatability and adjusting the viscosity of the coating liquid during the formation of an adhesive, and examples thereof include butanediol diglycidyl ether.

Furthermore, a volatile solvent may also be added for the purpose of adjusting the viscosity of the coating liquid. Examples of the solvent that may be used in the preparation of the coating liquid composition include ketone-based solvents such as methyl ethyl ketone and methyl isobutyl ketone; and ester-based solvents such as ethyl acetate and isobutyl acetate.

In addition, an adhesion strengthening agent such as a silane coupling agent may be added for the purpose of strengthening the adhesion to the base material, and a polyfunctional epoxy compound may be added for the purpose of increasing the crosslinking density and enhancing heat resistance.

It is also preferable to further add a filler such as silica, calcium carbonate or barium sulfate, to the epoxy resin layer. The addition of a filler allows an increase in the hardness of the resin layer, and suppression of the penetration of steam and a disinfectant liquid into the outer resin layer, so that heat resistance and chemical resistance are enhanced.

The epoxy resin layer is formed by applying a coating liquid for the formation of an epoxy resin layer prepared by dissolving or dispersing an epoxy resin and a curing agent as essential components, as well as the components mentioned above as necessary, in an appropriate solvent, on the surface of the particular polymer layer, and drying the coating liquid. For the purpose of accelerating the crosslinking of the epoxy resin, the resin layer may be cured by a heating treatment, and the heating is preferably carried out under the conditions of a temperature in the range of normal temperature (25° C.) to 135° C., and more preferably 60° C. to 100° C., in accordance with the main agent or curing agent used.

The coating liquid for the formation of an epoxy resin layer may be applied by an appropriate method in accordance with the viscosity of the coating liquid. If the coating liquid has low viscosity, it is preferable to employ a method of impregnating a brush or a paint brush with the coating liquid and thereby applying the coating liquid. If the coating liquid has high viscosity, a method of adhering the coating liquid in advance to an object in a stick form and thereby applying the coating liquid, may be employed. Furthermore, a method of applying the coating liquid by ejecting the coating liquid from a syringe may also be employed.

There are no particular limitations on the thickness of the epoxy resin layer, and the thickness may be appropriately selected based on the site of application or the purpose of use. From the viewpoint of the effects of the invention, the thickness of the epoxy resin layer is preferably from 50 μm to 600 μm.

(Formation of Thread Winding Structure)

When a thread winding structure formed by fastening the joint between members by winding with a thread, is used for the partial structure according to the invention, after this thread winding structure has been formed, an epoxy resin layer may be formed so as to cover the thread winding structure.

The thread used in the formation of the thread winding structure may be any thread that is capable of withstanding the sterilization treatment conditions in an autoclave or the like.

Specific examples that may be used include a monofilament or a twisted yarn of a fiber formed from a heat resistant resin such as polyphenylene sulfide (PPS), polyether ether ketone (PEEK) or polyether imide (PEI); and a twisted yarn formed from a fiber having excellent heat resistance, such as an aramid fiber, a polyallylate fiber, a polyparaphenylenebenzobisoxazole fiber, or a carbon fiber. From the viewpoint of durability, a polyphenylene sulfide fiber is preferred.

The thread may be a twisted yarn or a monofilament, but from the viewpoint of having satisfactory penetrability of the adhesive into the thread winding unit, a monofilament (single fiber) is more preferred.

A suitable thickness of the thread is in the range of 30 μm to 150 μm, and more preferably in the range of 50 μm to 100 μm.

Upon the joining of members, the peripheries of the joint ends of the members are fastened by winding a thread, and thus a thread winding structure is formed, but there are no particular limitations on the method of fastening by winding a thread. However, from the viewpoint of the reliability of the joint portion, it is preferable to achieve the winding at a density as high as possible, and when it is considered that the endoscope is inserted into a human body and used, it is preferable that the surface has less surface irregularity. Therefore, from this point of view, it is preferable to adopt helical winding, by which it is possible to fasten by winding the thread in one layer at a high density, so that the thread is wound without overlapping one on another.

Furthermore, in the case of covering the thread winding structure with an epoxy resin layer, with regard to a preferred range of thickness of the epoxy resin layer, the thickness of the epoxy resin layer is preferably equal to or greater than the diameter of the wound thread, and is preferably a thickness which covers the thread winding structure and does not allow the wound thread to be exposed. Furthermore, the thickness is preferably larger than the outermost circumference of the wound thread by at least 10 μm or more, and when it is considered preferable that a smooth outer surface is maintained and the thickness is small, the thickness is preferably smaller than the outermost circumference of the wound thread plus 30 μm.

According to the invention, the surface of a metallic member is effectively protected, and at the same time, the metallic member surface and the epoxy resin layer are firmly adhered, all by the function of the particular polymer layer. Therefore, when the particular polymer layer is used in a connection section or a joint portion, the physical strength related to the joint or fixing is augmented, and during the washing of the endoscope, scratching is prevented, and also, an enhancement of heat resistance and chemical resistance is promoted during a sterilization treatment. Even in the case where the particular polymer layer is used at a connection area with a movable unit, a good balance between adhesiveness and shape conformability is achieved, and excellent durability is obtained.

Accordingly, it can be said that the effects of the invention are conspicuous when the invention is applied to a structure using a metallic member containing iron, such as a rigid endoscope.

In this specification, an example of having a thread winding structure at a joint between a rigid section, which is a metallic tube made of SUS, and a curved section covered with an elastomer, has been described in detail, but the invention is not intended to be limited to this, and a highly reliable joint portion with excellent protectiveness may be formed by applying the thread winding structure to any area where a metallic member and an organic material are joined.

For example, in the case of fixing, to a metallic member, a member that is to be fixed to the metallic member, the fixing process may be carried out such that a particular polymer layer is formed on the surface of the metallic member, an epoxy resin that is capable of forming an epoxy resin layer is disposed on the surface of the particular polymer layer, a member to be fixed is disposed to be in contact with the epoxy resin, and then the epoxy resin layer is cured. Here, there are no particular limitations on the member to be fixed, and the member to be fixed may be, for example, any of a resin material molded product such as a soft casing, and a coating member such as a resin sheet.

Hereinafter, exemplary embodiments of the present invention will be explained.

<1> An endoscopic apparatus comprising a partial structure, wherein the partial structure includes:

a metallic member;

a polymer layer that contains a polymer including a vinylidene fluoride unit; and

an epoxy resin layer disposed on the polymer layer,

and wherein the polymer layer and the epoxy resin layer are disposed in this order on a surface of at least a part of the metallic member.

<2> The endoscopic apparatus of <1>, further comprising a second member that is fixed to the metallic member at the partial structure as a joint portion.

<3> The endoscopic apparatus of <1> or <2>, wherein the polymer layer and the epoxy resin layer extend to cover at least a part of the second member.

<4> The endoscopic apparatus of any one of <1> to <3>, wherein the polymer layer containing a polymer which includes a vinylidene fluoride unit is a layer that further contains a silane coupling agent.

<5> The endoscopic apparatus of any one of <1> to <4>, wherein the metallic member contains iron.

<6> The endoscopic apparatus of any one of <1> to <5>, wherein the polymer which includes a vinylidene fluoride unit is a polymer including a structural unit comprising a fluoroalkylene unit or a fluorovinyl ether unit as a copolymerization component. <7> The endoscopic apparatus of any one of <1> to <6>, wherein a thickness of the polymer layer containing a polymer which includes a vinylidene fluoride unit is from 1 μm to 50 μm, and a thickness of the epoxy resin layer is from 50 μm to 600 μm.

<8> A method of manufacturing an endoscopic apparatus having a partial structure formed by fixing, to a metallic member, a member that is to be fixed to the metallic member, the method comprising, in the recited order:

applying, on a surface of the metallic member, a composition for a formation of a polymer layer containing a polymer which includes a vinylidene fluoride unit, and a silane coupling agent, and drying the composition to thereby form a polymer layer;

applying, on a surface of the polymer layer, a composition for a formation of a resin layer containing an epoxy resin, to thereby form an epoxy resin coating liquid layer; and

bringing the member to be fixed, into contact with a surface of the epoxy resin coating liquid layer, and applying energy to cure the epoxy resin layer.

<9> A method of manufacturing an endoscopic apparatus including a partial structure at which a second member is fixed to a metallic member, the method comprising, in the recited order:

disposing the metallic member and the second member so as to abut each other;

applying a composition for a formation of a polymer layer to a surface of at least a part of the metallic member and a surface of at least a part of the second member at or around a joint between the metallic member and the second member, and drying to form a polymer layer, wherein the composition for a formation of a polymer layer contains a polymer including a vinylidene fluoride unit;

forming, by winding with a thread, a thread winding structure which fastens a surface of the polymer layer and fixes the metallic member and the second member to each other; and

applying an epoxy resin-containing composition for formation of a resin layer to a surface of the thread winding structure so as to cover the thread winding structure, and then applying energy to cure the epoxy resin layer.

There are no particular limitations on the joint portion according to the invention, as long as the joint portion is a region including an area in which an epoxy resin layer is formed on the surface of a metallic member, such as a joint section between a metallic rigid member and a flexible member, a joint portion between a metallic member and a tube-shaped flexible casing, or a coating section for the protection of the surface of a metallic member.

Specific examples of fixing modes include an aspect in which the mouth portion of a flexible tube that is inserted through the inside of the insertion unit of an endoscopic apparatus, is fixed to the body of the front tip section or the bent section of the insertion unit; an aspect in which a frame of an optical system such as the lens group disposed at the front tip of the insertion unit, is fixed to the body of the front tip section; an aspect in which the bent section that is inserted through a flexible tube is fixed to the body of the front tip section; an aspect of protecting and fixing a wiring or the like of an optical system such as CCD, which has been incorporated into the body of the front tip section; an aspect of fixing a metallic member to a flexible member in a rigid endoscope.

### EXAMPLES

Hereinafter, the invention will be specifically described by way of Examples, but the invention is not intended to be limited to the Examples, and various modifications, alterations and improvements may be made.

### Example 1

### Formation of Layer Containing Polymer which Includes Vinylidene Fluoride Unit

A coating liquid for the formation of a particular polymer layer having the following composition, containing a polymer which includes 40% by mole to 90% by mole of a vinylidene fluoride unit (trade name: G-902, manufactured by Daikin Industries, Ltd.), was prepared.

(Coating Liquid for Formation of Particular Polymer Layer)

| G-902 | 5 | g |
|---|---|---|
| (trade name, manufactured by Daikin | | |
| Industries, Ltd; raw fluororubber) | | |
| Carbon black | 1 | g |
| (manufactured by Tokai Carbon Co., Ltd.) | | |
| Triallyl isocyanurate | 0.8 | g |
| (manufactured by Nippon Kasei Chemical | | |
| Co., Ltd.; peroxide) | | |
| PERHEXA 25B | 0.3 | g |
| (trade name, manufactured by NOF | | |
| Corporation; co-crosslinking agent) | | |
| KBM-1003 | 1 | g |
| (trade name, manufactured by Shin-Etsu | | |
| Chemical Co., Ltd.; silane coupling agent) | | |
| Methyl ethyl ketone | 100 | ml |

A test piece made of 0.5t-SUS304 and having a width of 5 mm, was used as a metallic member, and the test piece was coated by immersing in the coating liquid for the formation of a particular polymer layer. The test piece was treated for 5 minutes in a hot nitrogen gas stream at 150° C., and thus a particular polymer layer having a thickness of 2 μm was formed.

(Formation of Epoxy Resin Layer)

Two sheets of a laminate formed by forming a particular polymer layer on the surface of a metallic member, were used, and EPO-TEK 353-DT (trade name, manufactured by Epoxy Technology, Inc.; epoxy adhesive) was applied on the surface of the particular polymer layer of one sheet of the laminate. The other sheet of the laminate was closely adhered to the surface of the applied layer such that the particular polymer layer was adjacent to the applied layer, and the assembly was heated at 80° C. for 30 minutes to cure the epoxy resin layer, so as to adhere the two sheets. Thus, an adhered sample (1) of Example 1 was produced. The thickness of the epoxy resin layer thus formed was 100 μm.

(Initial Peeling Strength Test)

For the sample (1) obtained in Example 1, the shear peeling strength was measured by the method according to JIS K 6850 (2008), and the 90° peeling strength was measured by the method according to JIS K 6854-1 (2008). The shear peeling strength was 12 N/mm², and the 90° peeling strength was 10 Nmm.

Thereby, it was found that the sample (1) obtained in Example 1 exhibits a sufficient peeling strength with a good balance in the stresses in various directions.

(Adhesion Durability Test 1: Resistance to Autoclaving)

The sample (1) obtained in Example 1 was placed in a small-sized gravity type autoclave and was treated with steam at 135° C. for one week and for two weeks. Thereafter, shear peeling strength was measured in the same manner as in the measurement of the initial peeling strength, and the value of the shear peeling strength was 10 N/mm² for both after one week and after two weeks. Even after a treatment by autoclaving for a long time, a decrease in the peeling strength was hardly observed.

(Adhesion Durability Test 2: Resistance to Hydrogen Peroxide)

The sample was subjected to immersion in aqueous hydrogen peroxide at a concentration of 30% maintained at 5° C. for one week and for two weeks. Thereafter, the shear peeling strength was measured in the same manner as in the measurement of the initial peeling strength, and the value obtained after one week was 9 N/mm², while the value obtained after two weeks was 8.1 N/mm². It was found that even after a treatment with hydrogen peroxide for a long time, practically sufficient peeling strength was maintained.

### Comparative Example 1

The test piece made of 0.5t-SUS304 and having a width of 5 mm, which was used in Example 1, was used; however, the particular polymer layer was not formed thereon, and the epoxy resin layer used in Example 1 was formed. Two sheets of the test pieces were adhered, with the epoxy resin layer interposed therebetween, and thus a sample (C1) of Comparative Example 1 was obtained.

The peeling strengths of the sample (C1) were measured in the same manner as in Example 1, and the shear peeling strength was 15 N/mm², while the 90° peeling strength was 2 N/mm². The 90° peeling strength was a practically insufficient value.

Thereby, it was found that the sample (C1) obtained in Comparative Example 2 was not suitable for the use in the sites that are subject to stress from an angle other than the shear stress, such as a bent section.

With regard to the resistance to autoclaving, the value obtained after one week was 2 N/mm², and after two weeks, the sample went through natural peeling. From this, it was found that the sample (C1) obtained in Comparative Example 1 was inferior in the resistance to autoclaving. With regard to the resistance to hydrogen peroxide, the value obtained after one week was 4 N/mm², and the value obtained after two weeks was 2 N/mm². It was found that the sample (C1) was not durable against the sterilization treatment using hydrogen peroxide.

### Comparative Example 2

The test piece made of 0.5t-SUS304 and having a width of 5 mm, which was used in Example 1, was used; however, the particular polymer layer was not formed thereon, and an epoxy resin layer was formed using a coating liquid obtained by adding a silane coupling agent (trade name: KBM-403, manufactured by Shin-Etsu Chemical Co., Ltd.) at a proportion of 2% to the coating liquid for the formation of an epoxy resin layer used in Example 1. Two sheets of the test pieces were adhered, with the epoxy resin layer interposed therebetween, and thus a sample (C2) of Comparative Example 2 was obtained.

The peeling strengths of the sample (C2) were measured in the same manner as in Example 1, and the shear peeling strength was 20 N/mm², while the 90° peeling strength was 3 N/mm². The 90° peeling strength was a practically insufficient value.

Thereby, it was found that the sample (C2) obtained in Comparative Example 2 was not suitable for the use in the sites that are subject to stress from an angle other than the shear stress, such as a bent section.

With regard to the resistance to autoclaving, the values obtained after one week and after two weeks were all 10 N/mm², and from this, it was found that the sample (C2) obtained in Comparative Example 2 had satisfactory resistance to autoclaving. With regard to the resistance to hydrogen peroxide, the value obtained after one week was 7 N/mm², and the value obtained after two weeks was 3 N/mm². It was found that the sample (C2) was not durable against the sterilization treatment using hydrogen peroxide.

### Example 2

A curved section core material formed from a metallic articulated tube and a reticulated tube was joined with a stainless steel tube of the body of a rigid endoscope, and then the coating liquid for the formation of a particular polymer layer used in Example 1 was applied by brush coating and dried. Subsequently, the coated assembly was subjected to a partial heating treatment using an infrared concentration heating device. Then, a flexible casing was applied on the treated surface of the curved section care material.

Subsequently, a monofilament of polyphenylsulfone having a diameter of 100 μm (trade name: KPS, manufactured by Kureha Gohsen Co., Ltd.) was wound to cover the joint section, and thereby the tube of the curved section (flexible casing) was fixed by fastening by winding a thread. Furthermore, a mixture obtained by mixing 100 parts of a main agent of EPO-TEK 353-DT (trade name, manufactured by Epoxy Technology, Inc.; epoxy adhesive), which is an epoxy resin, with 10 parts of a curing agent, 20 parts of calcium carbonate (manufactured by Shiraishi Kogyo) and 1 part of carbon black (manufactured by Tokai Carbon Co., Ltd.), was applied and cured above the thread winding structure. Thus, the bobbin structure was formed by coating.

The connection of the rigid stainless steel tube and the curved section (sample B) thus produced was used to perform a durability test by the method described below.

(Aqueous Hydrogen Peroxide Immersion Test)

The specimen thus obtained was submerged in a 30% aqueous hydrogen peroxide for two weeks, and then the specimen was removed, washed and dried. An observation of the external appearance was carried out. Furthermore, the tube tip was immersed in water to thereby pressurize the inside of the tube, and thus the presence or absence of leakage was investigated.

(Autoclave Durability Test)

The specimen was placed in an autoclave and was subjected to a treatment for 100 hours under a temperature condition of 135° C. The specimen was removed, and an observation of the external appearance was carried out. Furthermore, the tube tip was immersed in water to thereby pressurize the inside of the tube, and thus the presence or absence of leakage was investigated.

In the insertion tube for the rigid mirror attached to the curved section of the sample B according to the invention, there was no change in the external appearance, and leakage was not recognized after both the hydrogen peroxide test and the autoclave immersion test.

### Comparative Example 3

Use was made of a connection of a rigid stainless steel tube and a curved section [sample (2C)] which was produced in the same manner as in Example 2, except that the epoxy resin containing a silane coupling agent as used in Comparative Example 2 was used, and the particular polymer layer was not formed, and a durability test was carried out by the same method as that used in Example 2. As a result, no change in the external appearance was recognized, but air leakage was recognized after both the hydrogen peroxide treatment and the autoclave immersion treatment. Thus, it was found that defects had occurred in the epoxy resin layer.

According to the invention, even when an endoscopic apparatus having a metallic member is subjected to an autoclave sterilization treatment and a hydrogen peroxide plasma sterilization treatment, the occurrence of damage or deterioration in the resin layer at the joint portion between the metallic member and a flexible casing or between metallic members, is suppressed, and therefore, a medical endoscopic apparatus having excellent durability may be provided.

Furthermore, according to the invention, a method of manufacturing an endoscopic apparatus, which is capable of producing the medical endoscopic apparatus having excellent durability simply and easily, may be provided.

What is claimed is:

1. An endoscopic apparatus comprising a partial structure, wherein the partial structure includes:
a metallic member;
a polymer layer that contains a polymer including a vinylidene fluoride unit; and
an epoxy resin layer disposed on the polymer layer,
and wherein the polymer layer and the epoxy resin layer are disposed in this order on a surface of at least a part of the metallic member.

2. The endoscopic apparatus of claim 1, further comprising a second member that is fixed to the metallic member at the partial structure as a joint portion.

3. The endoscopic apparatus of claim 2, wherein the polymer layer and the epoxy resin layer extend to cover at least a part of the second member.

4. The endoscopic apparatus of claim 1, wherein the polymer layer containing a polymer which includes a vinylidene fluoride unit is a layer that further contains a silane coupling agent.

5. The endoscopic apparatus of claim 1, wherein the metallic member contains iron.

6. The endoscopic apparatus of claim 1, wherein the polymer which includes a vinylidene fluoride unit is a polymer including a structural unit comprising a fluoroalkylene unit or a fluorovinyl ether unit as a copolymerization component.

7. The endoscopic apparatus of claim 1, wherein a thickness of the polymer layer containing a polymer which includes a vinylidene fluoride unit is from 1 μm to 50 μm, and a thickness of the epoxy resin layer is from 50 μm to 600 μm.

8. A method of manufacturing an endoscopic apparatus having a partial structure formed by fixing, to a metallic member, a member that is to be fixed to the metallic member, the method comprising, in the recited order:
applying, on a surface of the metallic member, a composition for a formation of a polymer layer containing a polymer which includes a vinylidene fluoride unit, and a silane coupling agent, and drying the composition to thereby form a polymer layer;
applying, on a surface of the polymer layer, a composition for a formation of a resin layer containing an epoxy resin, to thereby form an epoxy resin coating liquid layer; and
bringing the member to be fixed, into contact with a surface of the epoxy resin coating liquid layer, and applying energy to cure the epoxy resin layer.

9. A method of manufacturing an endoscopic apparatus including a partial structure at which a second member is fixed to a metallic member, the method comprising, in the recited order:
disposing the metallic member and the second member so as to abut each other;
applying a composition for a formation of a polymer layer to a surface of at least a part of the metallic member and a surface of at least a part of the second member at or around a joint between the metallic member and the second member, and drying to form a polymer layer, wherein the composition for a formation of a polymer layer contains a polymer including a vinylidene fluoride unit;
forming, by winding with a thread, a thread winding structure which fastens a surface of the polymer layer and fixes the metallic member and the second member to each other; and
applying an epoxy resin-containing composition for formation of a resin layer to a surface of the thread winding structure so as to cover the thread winding structure, and then applying energy to cure the epoxy resin layer.
